(19) United States
(12) Patent Application Publication
(10) Pub. No.: US 2006/0090759 A1
(43) Pub. Date: May 4, 2006

(54) Conserver design for a therapeutic breathing gas system

(76) Inventors: Lawrence Howes, Santa Barbara, CA (US); Geoffrey Frank Deane, Goleta, CA (US); Brenton Alan Taylor, Kenwood, CA (US); Chung-Ming Li, Goleta, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/170,743

(22) Filed: Jun. 28, 2005

Related U.S. Application Data
(60) Provisional application No. 60/583,044, filed on Jun. 28, 2004.

(51) Int. Cl. A61M 16/00; A62B 7/04

(52) U.S. Cl. 128/204.21; 128/204.26

(57) ABSTRACT

In an embodiment, the invention is an improved interface to a conserver for therapeutic gas delivery. The improved circuit allows for fine control of the frequency response of a feedback element, which permits a high gain interface. The result is excellent compensation of transducer drift with little distortion of the frequencies of interest for breath detection, even for low breath pressure scenarios.

The present application claims priority benefit under 35 U.S.C. § 119(e) from U.S. Provisional Application No. 60/583,044, filed Jun. 28, 2004, entitled A CONSERVER DESIGN FOR A THERAPEUTIC BREATHING GAS SYSTEM, the entirety of which is hereby incorporated herein by reference.

### RELATED APPLICATIONS

The present application is related to pending U.S. patent application Ser. No. 11/147,409, filed Jun. 6, 2005, entitled SYSTEMS AND METHODS FOR DELIVERING THERAPEUTIC GAS TO PATIENTS, which is hereby incorporated herein in its entirety by reference.

### BACKGROUND OF THE INVENTION

1. Field of the Invention

The present invention relates to breath sensing devices, and is particularly applicable to breath sensing devices used in conjunction with therapeutic gas delivery systems such as oxygen concentrators.

2. Description of the Related Art

The application of oxygen concentrators for therapeutic use is known and many variants of such devices exist. A particularly useful class of oxygen concentrators is designed to be portable, allowing users to move about and to travel for extended periods without the need to carry a supply of stored oxygen. Such portable concentrators must be small and light to be effective. Concentrators in general are implicitly limited in terms of the rate at which they can deliver oxygen to the patient, but benefit because they are only duration-limited by their access to electric power. To make the portable concentrators small and light, the rate at which oxygen is concentrated by the device is further restricted. However, use of a device called a conserver, which is placed in the product line between the concentrator and the patient, mitigates this limitation.

The conserver, many designs of which are known in the art, senses a patient's breath demand, and responds by delivering a volume of oxygen-rich gas (known as a bolus) to the patient. This bolus, which is significantly less than the total volume of a typical inhalation, is entrained in the breath's air intake, and mixes with the air, eventually reaching the lungs, esophagus, and respiratory cavities (nose and mouth). Approximately half of an inspiration enters the lungs, where oxygen is absorbed. Elevated oxygen concentrations in this volume result in greater transfer of the gas to the blood, which enhances the health of the patient. Because the lungs can only make use of oxygen in the volume that reaches them, it is important that the bolus be delivered during the portion of an inhalation that actually reaches the lungs. As this is typically the first 50% of a breath, it is clear that the bolus must be delivered quickly, requiring that the bolus delivery start as rapidly as possible after the start of the patient's breath.

Quick delivery of the bolus generally allows smaller boluses to be delivered while still satisfying the patient's need for oxygen. Thus, the conserver delivers an effective therapeutic amount of oxygen in relatively small, short bursts, constituting a more efficient use of the concentrated product gas. This allows for the design of small, lightweight concentrators that are equally effective as large continuous flow gas supplies.

However, it is desirable to optimize the conserver's efficacy during a wide range of patient activities, including rest and sleep states. Thus, it is desirable that the conserver can accommodate a wide variety of breath conditions. The conserver's sensitivity, or the magnitude of the threshold inhalation vacuum pressure (typically sensed through a nasal cannula), is typically the key parameter that is used to trigger a bolus delivery. In order to reduce false triggers (bolus delivery when no breath has occurred), breath detection, which is accomplished by measuring inhalation vacuum pressure, is typically set to a threshold level that corresponds to normal daytime breathing and activity patterns, referred to hereafter as low sensitivity operation.

Many conserver designs include a pressure transducer and an electronic transducer interface. One such transducer and electronic interface are described in U.S. Pat. No. 6,810,877, entitled HIGH SENSITIVITY PRESSURE SWITCH, herein incorporated by reference in its entirety. In the '877 patent, the transducer is subjected to requirements that correspond to daytime activities and a physical configuration where the transducer is close to the patient. Thus, the choice of transducer allows for a circuit gain of less than 10,000. As such, the techniques described in the '877 patent yield good performance for the low sensitivity regime.

However, this level of performance may not be sensitive enough to reliably detect breathing for rest or sleep conditions. If the trigger pressure is too high (sensitivity too low), the conserver does not recognize a breath until a significant portion of it has already been inspired, thereby reducing the efficacy of the delivered bolus.

Moreover, in some conserver applications the transducer may be exposed to pressures many orders of magnitude greater than the measured inspiratory pressure range. A wider range transducer may be desirable in these cases in order to avoid pressure-induced damage to the transducer. In this case, the transducer signal's gain can be greater than 50,000.

While the basic circuit in the '877 patent represents improvements in conserver or breath detection sensitivity, certain preferred embodiments of the present invention describe improvements to the circuit of the '877 patent that further extend its use to high gain transducer circuits and to higher sensitivity applications, such as nighttime operation.

### SUMMARY OF THE INVENTION

In one aspect, the preferred embodiments of the present invention provide an improved breath pressure measurement device. The device comprises a pressure transducer for detecting inspiratory breath pressure, and an electronic interface to the transducer containing a delayed feedback component, wherein the delayed feedback component can be adjusted under user control. In one embodiment, the magnitude of the feedback component can be selected from predetermined amounts by a controller. In a further embodiment, the predetermined magnitudes are selected by the controller switching between combinations of attenuation networks. In yet another embodiment, the feedback component can be switched on and off in a continuous manner by a pulse width modulation signal supplied from a controller to a switching device. The duty cycle of the pulse width modulation signal can varied adaptively by the controller to achieve proper sensitivity over a wide range of patient activity levels and breathing patterns. Furthermore, the feedback component can be switched off entirely during times where negative feedback is not desired.

In another aspect, the preferred embodiments of the present invention provide an improved breath pressure measurement device, which includes a pressure transducer configured to detect inspiratory breath pressure and to output an electric signal with an amplitude proportional to the pressure level, an amplifier configured to amplify the output of the pressure transducer, a comparator configured to compare an output of the amplifier with a predetermined threshold, and a feedback circuit having an input coupled to an output of the comparator and configured to generate a bias voltage for the amplifier, wherein the frequency response of the feedback circuit is adjustable.

In yet another aspect, the preferred embodiments of the present invention provide an apparatus for controlling a conserver valve. The apparatus comprises a breath sensor and a programmable controller. The breath sensor produces a signal in response to sensing a breath. The programmable controller comprises a control circuit which amplifies the breath sensor signal, and a circuit controller which alters the response of the control circuit to the breath sensor. The programmable controller produces a valve control signal which controls the valve.

In yet another aspect, the preferred embodiments of the present invention provide an apparatus for controlling a conserver valve. The apparatus comprises a breath sensor which produces a signal in response to sensing a breath, and a programmable controller. The programmable controller includes a control circuit, which amplifies the sensor signal, and a feedback circuit having a frequency response dependent on a time constant of the feedback circuit. The programmable controller produces a valve control signal which controls the valve. The programmable controller further includes a circuit controller which alters the time constant of the feedback circuit to alter the response of the control circuit to the breath sensor.

In yet another aspect, the preferred embodiments of the present invention provide a method of controlling a conserver valve. The method includes producing a valve control signal in response to detection of breath, using the control signal to control the valve, and adjusting the valve control signal. The adjusting comprises operating a circuit in at least two modes, where the circuit is more sensitive to breaths in one of the modes than in another of the modes.

In yet another aspect, the preferred embodiments of the present invention provide a method of controlling a conserver valve. The method includes producing a valve control signal in response to detection of breath, using the control signal to control the valve, and adjusting the valve control signal using a valve control circuit. The adjusting comprises operating the valve control circuit such that the sensitivity of the circuit to breaths varies over time.

For purposes of summarizing the invention, certain aspects, advantages, and novel features of the invention have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1 is a block diagram of a therapeutic gas delivery system, according to an embodiment of the invention.

FIG. 2 is a graphic illustration showing the relationship between the timing of a bolus delivery during an inspiratory cycle and the efficacy of the gas delivered.

FIG. 3 is a graphic illustration showing the pressure profiles of exemplary inspiratory cycles of a patient's breath during normal activity and during sleep.

FIG. 4 is a block diagram of an embodiment of a conserver circuit.

FIG. 5 is a block diagram of an embodiment of the conserver circuit allowing for adjustment of the amount of feedback.

FIG. 6 is a block diagram of another embodiment of the conserver circuit allowing for adjustment of the amount of feedback.

FIG. 7A is a graphical illustration showing individual bolus deliveries as a function of time.

FIG. 7B is a graphical illustration showing variations in bolus delivery triggering parameters as a function of the elapsed time between successive bolus deliveries.

### DETAILED DESCRIPTION OF THE PREFERRED EMBODIMENT

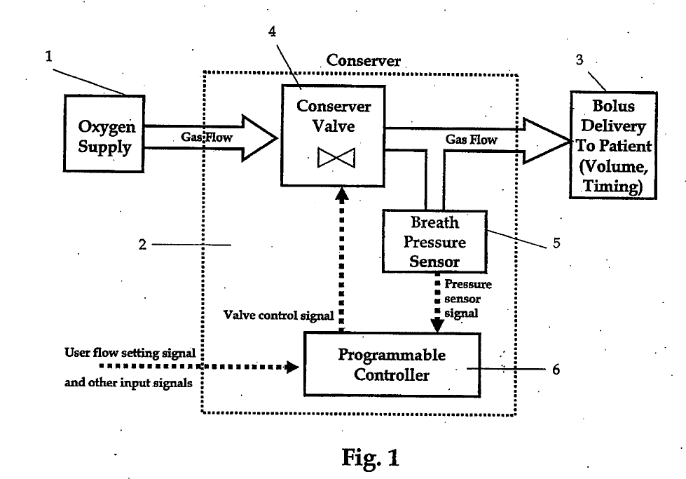

In a preferred embodiment of the present invention, an improved breath sensing device is incorporated as part of a therapeutic gas delivery system as illustrated in FIG. 1. The system generally includes an oxygen source 1 and a conserving device 2 for controlling the delivery of the oxygen to a patient 3. The oxygen source 1 can be an oxygen concentrator, a high-pressure oxygen tank, or any other device that supplies oxygen. One embodiment of the oxygen source 1 is described in U.S. Application Publication No. 20050072298, which is hereby incorporated by reference in its entirety.

As shown in FIG. 1, the conserving device 2 has a bolus delivery element 4, a breath sensor 5, and a programmable controller 6. The bolus delivery element 4 can include valves of the appropriate type and function. The breath sensor 5 is preferably a breath pressure sensor such as a transducer capable of detecting and measuring inspiratory breath pressure and transmitting signals to the programmable controller 6.

The programmable controller 6 includes an electronic conserver circuit and a circuit controller or microprocessor capable of determining the bolus volume and bolus timing based on the signals received from the breath sensor 5. In one implementation, the controller 6 determines the bolus volume by controlling how long the delivery valve 4 is kept open in each delivery, and controls the timing of the bolus by determining at which times the valve 4 is opened.

The desired functionality of the therapeutic gas delivery system includes the ability to measure inspiratory breath pressure and to control the open timing of the delivery valve, thereby controlling the volume of the bolus. In certain embodiments, the system is configured to address difficulties and problems associated with delivering therapeutic gas to a patient during sleep.

The efficacy of elevating oxygen concentrations in the lungs is generally known to relate to how much oxygen is delivered in early (alveolar) inspiration. While the exact fraction of inspired gas may vary from patient to patient, in general, the bolus volume delivered during the first half of an inspiratory cycle is more significant in oxygenating the patient. Thus, conserving devices 2 are preferably designed to deliver pulses of oxygen to the patient 3 during the very early stages of each inspiratory cycle.

Typically, the conserving device 2 triggers a bolus delivery when it detects a predetermined inspiratory pressure from the breath sensor 5. Thus, the term “threshold pressure” generally refers to the sensed inspiratory pressure at which a bolus delivery is triggered. In general, it is preferable to set the threshold pressure as high as possible to avoid triggering a bolus delivery based on false breath detection due to electrical signal noise or pressure noise in the cannula caused by patient activities. However, too high a setting can also render the therapy ineffective.

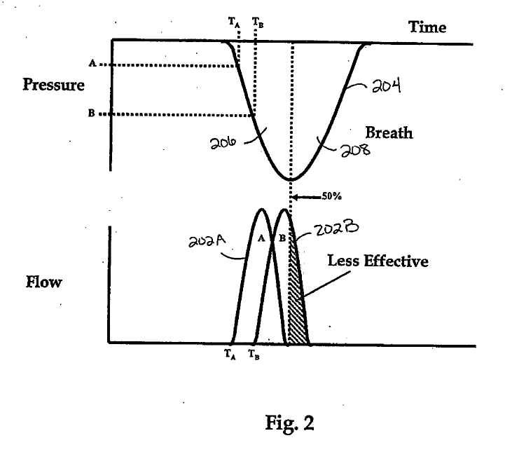

FIG. 2 is a graphic illustration of the relationship between the threshold pressure setting and the efficacy of the gas delivered. As shown in FIG. 2, the bolus delivery profiles 202A, 202B for two different threshold pressure settings TA and TB are correlated to a pressure profile 204 of a patient's inspiratory cycle. The pressure profile 204 comprises a first half 206 and a second half 208.

Threshold pressure level TA triggers delivery early enough to allow for full bolus delivery 202A in the first half 206 of the inspiratory cycle. Threshold pressure level TB, however, causes delivery of a significant portion of the bolus 202B in the second half 208 of the inspiratory cycle, and thus is not as effective. Accordingly, when the threshold pressure level is set too high relative to the inspiratory pressure of the very early stages of an inspiration cycle, a significant portion of the bolus is likely to be delivered during the second half of the inspiratory cycle 208, which renders the therapy less effective.

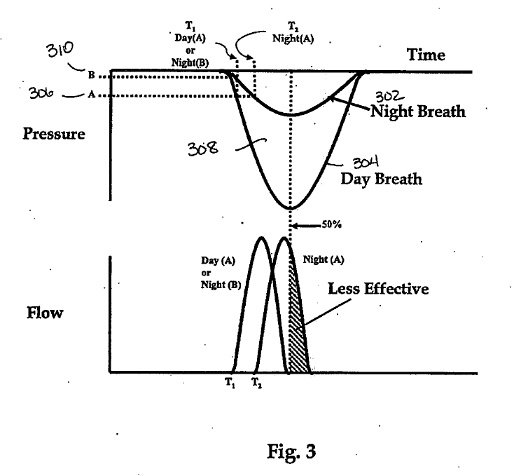

Problems associated with high threshold pressure settings are particularly apparent in conventional gas delivery systems when the patient is asleep or in a state of inactivity. As shown in FIG. 3, an inspiratory pressure profile 302 of a patient's breath during sleep may be much shallower than an inspiratory profile 304 of the patient's breath during normal activity. Thus, a threshold pressure value TA 306, which is effective during normal day activity, may be ineffective at night when the patient is asleep.

During sleep when the breaths are often shallower, the threshold pressure TA may not be reached sufficiently early in the inspiratory cycle 302 to allow a significant portion of the bolus to be delivered in a first half 308 of the cycle. FIG. 3 shows that a night response to threshold pressure TB 310 is equivalent to the day response to threshold pressure TA 306, although it is understood that the night bolus timing and volume do not have to correspond to the day bolus to be effective.

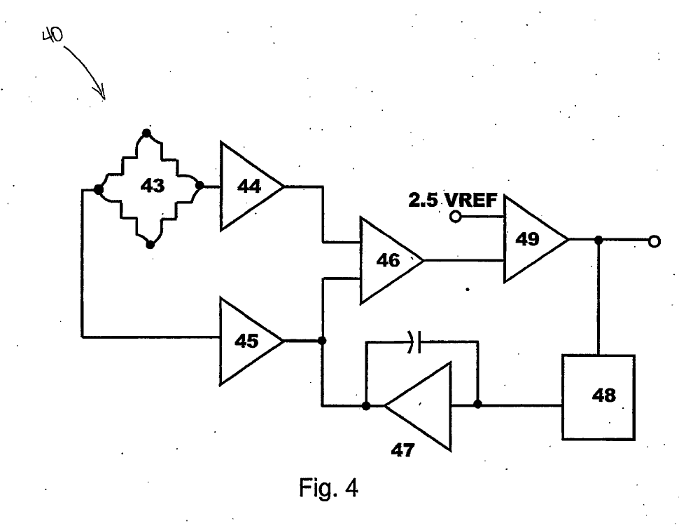

FIG. 4 is a simplified block diagram of an embodiment of a control circuit or conserver circuit 40 designed to improve breath sensing capabilities. The circuit 40 comprises a pressure transducer 43, which is an electronic interface to the breath pressure sensor 5, an instrumentation amplifier including elements 44, 45, 46, a feedback element 47, an initialization element 48, and a comparator 49.

The transducer 43 connects differentially to the instrumentation amplifier 44, 45, 46. Because many portable concentrators are battery powered, the circuit 40 is typically powered by a single ended supply. The circuit 40 used in a preferred embodiment is powered by 5 volts. Thus, the preferential midpoint or zero is 2.5 volts. Therefore, the transducer 43 is biased such that zero signal is 2.5 VDC. The output of the instrumentation amplifier 44, 45, 46 is compared to 2.5 VDC, such that a breath signal exceeds 2.5 VDC and causes the output of the comparator 49 to become positive, indicating that a breath has taken place.

In circuit 40, the transducer 43 produces voltages in response to a breath that require a gain of about 7000 in the instrumentation amplifier 44, 45, 46 to create usable signals. At this gain any significant drift in the zero point of the transducer 43 causes the amplifier 44, 45, 46 to saturate and the amplifier output to reach its maximum value, which renders breath signals undetectable. Pressure transducers 43 are subject to temperature drift as well as other errors over time that, if not compensated for, can make the circuit 40 unusable.

The feedback element 47 adjusts for drift of the zero point. The feedback element 47 is designed such that it has a very large time constant. Effectively, very slow changes to feedback element 47 are fed back to one terminal of the amplifier 46. Since drift takes place over minutes, changes in the zero point of the transducer 43 are subtracted from the breath signal before the high gain stage, but higher frequency signals such as the breath waveform are not fed back. The resulting gain of the amplifier 44, 45, 46 is very high for waveforms with frequencies of less than one hertz and above, and zero for slow changes to the zero bias point.

Since the time constant of the feedback element 47 is long, in order to allow for breath detection quickly when the conserver 2 is powered on, the initialization element 48 is included. The initialization element 48 disables the feedback until the capacitive component of the feedback element 47 is fully charged.

The circuit 40 described above works well for some conserver configurations during normal daytime operation. However, two goals of the present design may require higher performance from the conserver interface than can be achieved by the circuit 40.

First, an embodiment of the invention is designed to allow the pressure transducer 43 to be exposed to elevated supply pressures, and thus uses the transducer 43 with a wider range. Therefore, the actual signals produced by a breath can be as much as 10 times smaller than the signals in the circuit 40. Exacerbating this difficult requirement, an embodiment of the invention also works for low inspiratory pressure signals, such as those generated during shallow breathing while asleep, where the breath pressure signal can be much smaller than daytime operation. Thus, the amplifier gain for an embodiment of the invention is in the range of approximately 50 to approximately 100,000. This high gain significantly changes the effectiveness of the feedback element 47. In addition, such high gains cause any initial imbalances in the pressure transducers 43 to cause a high offset. In an embodiment, the high offset is advantageously zeroed without requiring manual offset adjustment of the circuit during initial setup.

Feedback element 47 relies on an RC time constant to discriminate between slow drift of the zero point and frequencies of interest for breath detection. However, practical values of R and C cannot be infinite, so there is a finite roll off for any RC element. Some signal in the frequency range of interest, including the breath signal itself, passes through feedback element 47. In the conserver circuit 40, the attenuated higher frequency signal fed back through feedback element 47 may be enough to cancel out the desired signal, particularly for shallow breath scenarios. Yet the drift adjustment benefit of the feedback element 47 is even more important for higher gain.

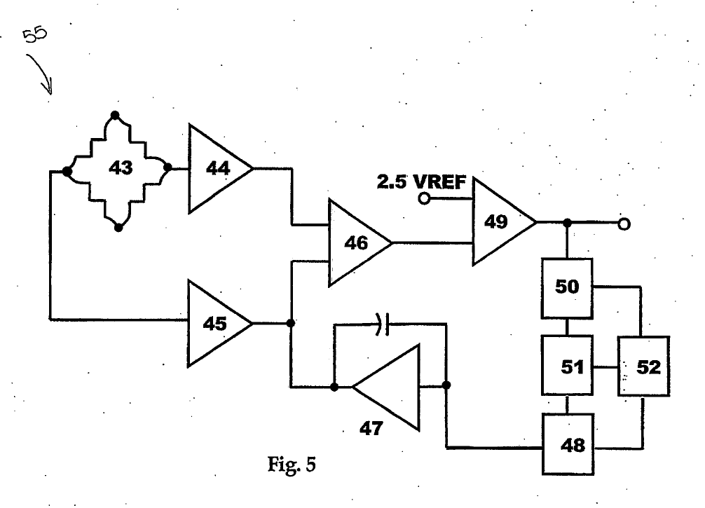

An embodiment of the invention, illustrated in FIG. 5, improves the conserver circuit design to solve the problem of signal cancellation through the feedback element 47 by allowing for adjustment of the amount of feedback. As shown in FIG. 5, a control circuit or conserver circuit 55 comprises the pressure transducer 43, the instrumentation amplifier 44, 45, 46, the feedback element 47, the initialization element 48, and the comparator 49. The circuit 55 further comprises networks 50, 51 in the feedback loop between the output of the comparator 49 and the input of the feedback element 47, and a controller 52, which enables at least a part of networks 50, 51.

The controller 52 changes the time constant of the feedback element 47 depending on the breath signal, or breath timing by controlling the value of the networks 50, 51 that is included in the feedback loop. A skilled designer will see many approaches to changing the time constant of the feedback element 47. One implementation is to switch in different resistor networks with solid state or mechanical relays, controlled by the controller 52, thereby changing the RC time constant of the feedback element 47. Thus, more feedback can be allowed when the breath signal is strong, or more importantly, during time when no breath signal is expected.

Since the zero drift is relatively slow, it is possible to enable the feedback element 47 for drift cancellation at selected times, and then turn down or even turn off the feedback when a breath is expected. Since the zero offset correction will be held by the capacitor of feedback element 47, the drift correction will change very little if the feedback input is removed for short periods.

As can be seen, there is utility in adjusting the amount of feedback from maximum to zero and points in between. This can be accomplished by the embodiment illustrated in FIG. 5 by having as many switchable networks 50, 51 as desired. In some embodiments, high value resistors, such as, for example, 10 mega-ohms or more, are used to produce the long RC time constant.

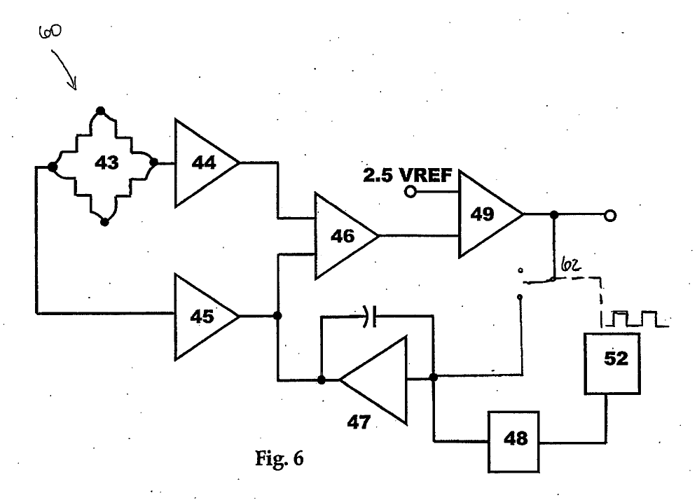

FIG. 6 illustrates another embodiment of an adjustable feedback element 47 for a control circuit or conserver circuit 60. As shown in FIG. 6, the circuit 60 comprises the pressure transducer 43, the instrumentation amplifier 44, 45, 46, the feedback element 47, the initialization element 48, and the comparator 49. The circuit 60 further comprises a switch 62 in the feedback loop between the output of the comparator 49 and the input of the feedback element 47, and the controller 52, which controls the switch 62.

Rather than change the RC time constant, the controller 52 controls the switch 62 to switch the feedback signal to the feedback element 47 on and off with pulse width modulation (PWM). Thus, a high duty cycle PWM signal turns the feedback element 47 on to accomplish the zeroing. Lowering the rate decreases the feedback. Thus, lowering the rate reduces the amount of feedback from a high frequency signal that can cancel the breath signal.

The capacitor of feedback element 47 provides the zero correction for breath detection scenarios even if the PWM duty cycle is very low or zero for part of the cycle. Thus, the PWM approach allows for effectively infinite resolution adjustment of the time constant of the feedback. A lower value resistor can also be used, since the amount of feedback can be reduced in a controllable fashion, and does not require a long RC time constant to avoid signal degradation at the breath signal frequencies. Also, a PWM duty cycle can be set to correct for inherent transducer offset present in the initial set-up as well.

After delivery of a bolus, there is a period before the next breath. The controller 52 can be programmed to not look for a breath during this “blind time”. During the blind time, the feedback can be turned on fully to perform zero drift correction. There can be a period after the blind time where a breath is possible, and the feedback is turned down to reduce breath signal cancellation. This increases the sensitivity of the breath detection by allowing lower pressure signals to be detected. The drift correction, in an embodiment of the present invention's circuit design, stays approximately constant for periods longer than typical breath periods when the feedback is turned down or off. Finally, if no breath is detected for a long period, or if previous breaths were very shallow, the feedback can be turned off entirely for some period, which maximizes the sensitivity for breath detection.

In certain preferred embodiments, the control circuit or conserver circuit 40, 50, 60 is located in the conserver 2, but it is understood by one of skill in the art that certain components of the circuit 40, 50, 60 may be located elsewhere, such as in the concentrator or oxygen supply 1.

Adaptive Control Responsive to Multiple Breath Parameters

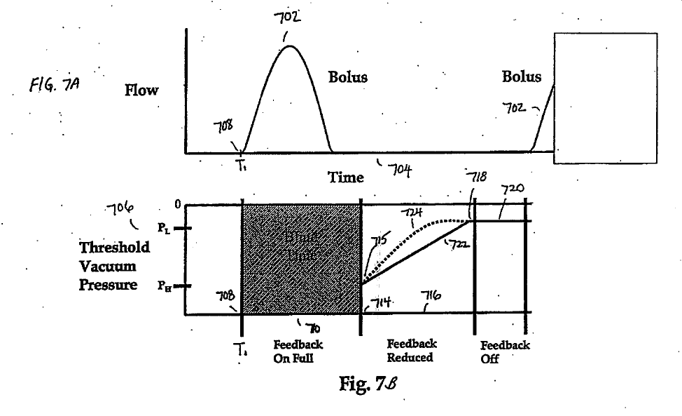

Preferably, the controller 6 is programmable to vary the bolus delivery to achieve various operational profiles. One operational profile is illustrated in FIGS. 7A and 7B.

The conserver design of one preferred embodiment of the present invention utilizes an adaptive control system to vary the bolus delivery in response to one or more breath parameters. This conserver design effectively addresses many of the above-described issues while improving immunity to false and ineffective triggers.

FIGS. 7A and 7B graphically illustrate the manner in which the adaptive control system of one preferred implementation varies the bolus delivery triggering parameters such as threshold pressure in accordance with the time elapsed between consecutive bolus deliveries. FIG. 7A shows individual bolus deliveries 702 as a function of time 704. FIG. 7B shows variations in bolus delivery triggering parameters 706 as a function of the elapsed time between successive bolus deliveries.

As shown, upon delivery of a bolus at T1 708, the control system alters the triggering parameters 706 by disabling the breath trigger for a blind time period 710 so that no bolus will be delivered during the blind time period 710. During the blind time period 710, the control system will not accept a breath trigger regardless of the breath pressure detected. The blind time period 710 can be in the range of about 0.5-3.0 seconds, preferably about 1.5 seconds. Upon the end 714 of the blind time period 710, the controller alters the triggering parameters by adjusting to a substantially noise immune, high threshold pressure level PH 715. If no breath is detected at this high threshold pressure level PH 715, the control system ramps the trigger sensitivity over a ramp time period 716 by gradually lowering the threshold pressure level until the threshold pressure reaches PL 718. The ramp time period 716 is preferably about 1-2 seconds. After the ramp time period 716, if no breath is detected even at the low threshold pressure level PL (high sensitivity) after a wait time period 720, typically about 2-3 seconds, a bolus is auto-fired. It will be appreciated that any suitable curve may be used such as the linear ramp 722 as shown in FIG. 7B. The inventors have found an exponential ramp 724 is effective as well.

As an example of the adaptive control system illustrated by the graphs of FIGS. 7A and 7B, for a typical oxygen patient breathing about 15 times per minute, a new inspiratory cycle is initiated every 4 seconds. After a bolus is delivered, the conserver spends the next 1.5 seconds blind, during which time all breath detection sensor input is ignored. After the blind period, the threshold vacuum pressure may be initially set at about 0.30 cm of water. Because the anticipated breathing period is 4.0 seconds (calculated from average breathing rates), the threshold pressure is controllably decreased over the next 2.25 seconds (1.5-3.75 seconds from last bolus) until it reaches a higher sensitivity level (lower threshold pressure) of about 0.08 cm of water. If, after an additional 2.75 seconds (6.5 seconds from last bolus) no breath has been detected, a bolus is automatically delivered.

Although a preferred application of the embodiments is for an oxygen conserver, there are other applications of the embodiments, such as sleep apnea devices.

While certain embodiments of the inventions have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A breath pressure measurement device, comprising:
a pressure transducer for detecting breath pressure; and
an electronic interface to the transducer containing a delayed feedback component, wherein the delayed feedback component may be adjusted under user control.

2. The device of claim 1 wherein the magnitude of the feedback component may be selected from predetermined amounts by a controller.

3. The device of claim 2 wherein the predetermined magnitudes are selected by the controller switching between combinations of attenuation networks.

4. The device of claim 1 wherein the feedback component may be switched on and off in a continuous manner by means of a pulse width modulation signal supplied from a controller to a switching device.

5. The device of claim 4 wherein a duty cycle of the pulse width modulation signal is varied adaptively by the controller to achieve proper sensitivity over a wide range of patient activity levels and breath patterns.

6. The device of claim 4 wherein the feedback component may be switched off entirely during times where negative feedback is not desired.

7. A breath pressure measurement device comprising:
a pressure transducer configured to detect inspiratory breath pressure and to output an electric signal with an amplitude proportional to the pressure level;
an amplifier configured to amplify the output of the pressure transducer;
a comparator configured to compare an output of the amplifier with a predetermined threshold; and
a feedback circuit having an input coupled to an output of the comparator and configured to generate a bias voltage for the amplifier, wherein the frequency response of the feedback circuit is adjustable.

8. The breath pressure measurement device of claim 7 wherein the frequency response is adjusted by switching the input of the feedback circuit on and off in a continuous manner as determined by a pulse width modulation signal supplied from a controller to a switching device.

9. The breath pressure measurement device of claim 7 wherein the frequency response is adjusted by switching between combinations of attenuation networks.

10. An apparatus for controlling a conserver valve, comprising:
a breath sensor which produces a signal in response to sensing a breath, said breath sensor comprises a pressure transducer for detecting breath pressure; and
a programmable controller comprising:
a control circuit which amplifies the sensor signal, said control circuit comprising an electronic interface to the transducer containing a delayed feedback component, and wherein the delayed feedback component is adjustable; and
a circuit controller which alters the response of the control circuit to the breath sensor, said programmable controller producing a valve control signal which controls the valve.

11. The apparatus of claim 10 wherein a pulse width modulation signal supplied from the circuit controller controls a switching device to switch the feedback component on and off in a continuous manner.

12. The apparatus of claim 11 wherein a duty cycle of the pulse width modulation signal is varied adaptively by the circuit controller to achieve proper sensitivity over a wide range of patient activity levels and breath patterns.

13. The apparatus of claim 11 wherein the feedback component may be switched off entirely during times where negative feedback is not desired.

14. An apparatus for controlling a conserver valve, comprising:
a breath sensor which produces a signal in response to sensing a breath; and
a programmable controller comprising:
a control circuit which amplifies the sensor signal, said programmable controller producing a valve control signal which controls the valve, the control circuit comprising a feedback circuit having a frequency response dependent on a time constant of the feedback circuit; and
a circuit controller which alters the time constant of the feedback circuit to alter the response of the control circuit to the breath sensor.

15. The apparatus of claim 14 wherein the magnitude of the feedback circuit may be selected from predetermined amounts by the controller.

16. The apparatus of claim 15 wherein the predetermined magnitudes are selected by the circuit controller by switching between combinations of attenuation networks.

17. The apparatus of claim 16 wherein the attenuation networks are resistor networks.

18. A method of controlling a conserver valve, comprising:
producing a valve control signal in response to detection of breath;
using the control signal to control the valve; and
adjusting the valve control signal, said adjusting comprising operating a circuit in at least two modes, said circuit being more sensitive to breaths in one of said modes than in another of said modes, wherein the circuit comprises a delayed feedback component, and wherein adjusting the valve control signal adjusts the delayed feedback component.

19. The method of claim 18 further comprising detecting breath pressure.

20. The method of claim 19 wherein adjusting the delayed feedback component comprises selecting a magnitude of the delayed feedback component from predetermined amounts.

21. The method of claim 20 wherein the predetermined magnitudes are selected by switching between combinations of attenuation networks.

22. A method of controlling a conserver valve, comprising:
producing a valve control signal in response to detection of breath;
using the control signal to control the valve; and
adjusting the valve control signal using a valve control circuit, said adjusting comprising operating the valve control circuit such that the sensitivity of the circuit to breaths varies over time.

23. The method of claim 22 wherein the valve control circuit comprises a delayed feedback component, and adjusting the valve control signal comprises adjusting the delayed feedback component.

24. The method of claim 23 wherein adjusting the delayed feedback component comprises switching the delayed feedback component on and off in a continuous manner by means of a pulse width modulation signal.

25. The method of claim 24 wherein a duty cycle of the pulse width modulation signal is varies adaptively to achieve proper sensitivity over a wide range of patient activity levels and breath patterns.

26. The method of claim 24 wherein the delayed feedback component may be switched off entirely during times where negative feedback is not desired.
